(19) United States
(12) Patent Application Publication
(10) Pub. No.: US 2004/0092897 A1
(43) Pub. Date: May 13, 2004

(54) Hygienic absorbent article

(76) Inventors: Carlos Da Silva Macedo Jr., Sao Paulo (BR); Eduardo Cezar Andreo Aledo, Sao Paulo (BR); Altair De Paula Vitor, Sao Paulo (BR); Francisco J. Valdivia Hernandez, Sao Paulo (ES)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/399,287

(22) PCT Filed: Oct. 18, 2001
(86) PCT No.: PCT/BR01/00132

(51) Int. Cl. A61F 13/15; A61F 13/20

(52) U.S. Cl. 604/358; 604/385.01

(57) ABSTRACT

The present disclosure relates to a disposable hygienic absorbent article having a shape that has a compulsory loss of material reduced to zero associated with conventional manufacturing processes.

### FIELD OF THE INVENTION

The present invention relates to a disposable hygienic absorbent article, such as diapers, incontinence pads and the like. More particularly, the present invention refers a disposable hygienic absorbent article having a shape that has a compulsory loss of material reduced to zero associated with conventional manufacturing processes.

### BACKGROUND OF THE INVENTION

In the following expressions, reference is made to a disposable diaper only in order to facilitate the disclosure, not excluding from the scope of the invention other disposable absorbent articles, such as products suitable for incontinence, sanitary napkins or the like.

Generically, a disposable diaper is a disposable article of a multilayer kind comprising a fluid permeable layer on the inner side in touch with the user's body. In the reverse or outer side of the diaper, a fluid impermeable layer having a absorbent core generically flat and elongated shaped, disposed between said layers.

The fluid permeable layer is generically made of a soft and nonirritating material. According to the present state of the art, it can be a sheet of woven or non woven material natural (for example wood or cotton fibers) or artificial fibers (for example polyester or polypropylene, or a combination of both, a perforated plastic film, a porous or cross-linked foam and the like. The fluid permeable layer may be of known hydrofilicity, showing a tendency to remain dry, and may be a mono or multilayered material.

The fluid impermeable layer has the role to prevent the absorbed and retained fluid in the absorbent core from passing to the user' clothes or skin, being generically a thin polyethylene film. However, the fluid impermeable may also be permeable to vapor and, in such case, will be provided with small pores or made of a material which provides impermeability only to fluids. The absorbent core of known disposable absorbent articles is usually formed of a cellulose fiber pulp, often comprising super-absorbent polymers capable of forming a gel in contact with liquids as well. Other absorbent cores are also known such as papers, peat moss, artificial fibers, and foams and like.

Processes for preparing and cutting disposable hygienic absorbent articles and in particular disposable diapers from a continuous strip of base material are known in the art. The cuts are made by knives, ultrasound, laser or water under pressure and the like, in order to obtain diapers cut in strip's longitudinal or transversal direction.

Usually, a semi finished absorbent structure of the kind herein dealt with has greater outer borders than the final product, being thus necessary to cut in order to remove, reduce or shape some specific areas, such as the groin region or adjacent regions. The state-of-the-art disposable diapers, aiming at the adaptation to the user's anatomy, show in the groin region a relatively narrow width preceded by enlargements at the opposite ends forming sinuous contours after cutting the base material strip. The cut portions of such shaped regions are discarded, without reusing such cut material.

The disadvantages of previous art cutting processes, due to the curved side border shape of these diapers plus the disposition of the diaper in the base material strip's longitudinal direction, reside in the high compulsory loss of material, compromising noticeably the productivity.

### SUMMARY OF THE INVENTION

In accordance with the present invention there has been provided a disposable hygienic absorbent article comprising a fluid permeable layer, a fluid impermeable layer, an absorbent core disposed between said fluid permeable layer and the impermeable layer, the absorbent article having a longitudinal symmetry axis, a rear region having a transverse length adapted to the user's waist and a front region having a transverse length shorter than the transverse length of the rear region, an intermediate region having a pair opposite of side borders adjacent to said rear and front regions and at least an attachment means in the front region or in the rear region adapted to join the front region to the rear region around the user's body, wherein the side borders converge mutually and symmetrically from the rear region to the front region, defining a slope angle, as measured between said side border and the longitudinal symmetry axis of the absorbent article.

It is an advantage of the present invention that the disposable hygienic absorbent article has a shape that provides a compulsory loss of material reduced to zero associated with conventional manufacturing processes.

In the following text, it must be understood that the expression “compulsory loss reduced to zero” refers to maximizing the disposition of adjacent absorbent in a manufacturing process, reducing the waste between the absorbent article.

### BIEF DESCRIPTION OF THE INVENTION

The objects of the present invention are achieved through a disposable hygienic absorbent article of the invention comprising a geometrical shape, wherein an intermediate region having a pair opposite side borders adjacent to a rear and a front regions of the absorbent article converge mutually and symmetrically in relation to the longitudinal axis, from along or adjacent to said rear region to said front region, defining a slope angle, as measured between said side border and the longitudinal symmetry axis of the absorbent article.

As to the slope angle of the respective side border of the intermediate region, it is a value chosen between 5 and 75 as measured between said side border and the longitudinal symmetry axis of the absorbent article, the preferred slope angle being 20.

A ratio of a transverse length of the rear region to a transverse length of the front region is at least 2, being preferably 3. The ratio of a longitudinal length to the transverse length of the front region is between 0.6 and 1.3, the preferred value being equal to 1.0.

### BRIEF DESCRIPTION OF THE DRAWINGS

The present invention will be next described in more details, based on the following figures, which show:

FIG. 1—an upper plan view of the inner side of a first embodiment of the disposable hygienic absorbent article of the present invention;

FIG. 2—an upper plan view of the inner side of a second embodiment of the present invention;

FIG. 3—an upper plan view of a region of a multilayer continuous sheet having the absorbent articles of FIG. 1 disposed before being separated;

FIG. 4—an upper plan view of a region of another multilayer continuous sheet having the absorbent articles of FIG. 2 disposed before being separated; and

FIG. 5—an upper plan view of a region of a multilayer continuous sheet of another embodiment of the absorbent articles of the invention before being separated.

### DETAILED DESCRIPTION OF THE FIGURES

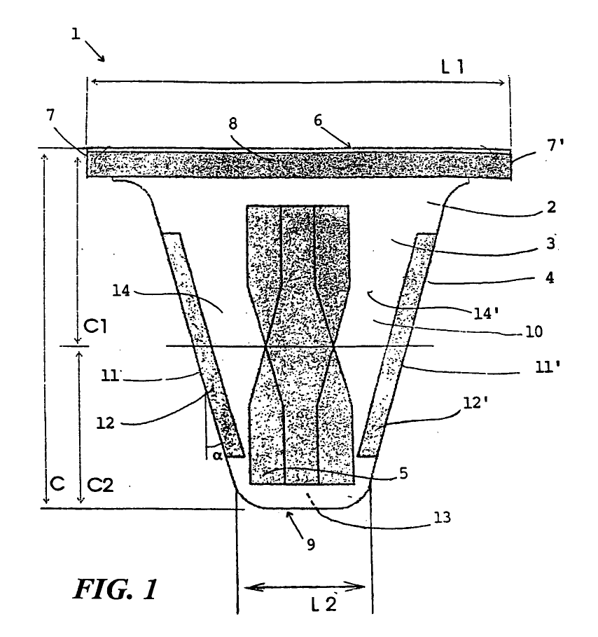

In a first embodiment of the invention as illustrated in FIG. 1, the disposable hygienic absorbent article indicated by 1 is a diaper for babies made in a unitary piece, having a geometrical shape substantially of a truncated cone section with an upwards base and a longitudinal length C.

Numeral 2 indicates a main body of the diaper 1, which consists of a fluid permeable layer 3 adapted to contact the user' body and a fluid impermeable layer 4. Between the layers 3 and 4 there is disposed an absorbent core 5 substantially planar shaped. The diaper has a rear region 6 of a transverse length L1 provided with an elastic stripe 8 that ends in a pair of attachment tabs 7 and 7′ forming a diaper's waist for adjustment to the user's waist.

The transverse length L1 of the rear region in the above mentioned example is obtained through the main body 2. However, the same transverse length L1 may be obtained through placing other additional materials, such as adhesive tapes and graft materials known in the state of the art by the term “extensions”.

Distally to the rear region 6 there is a front region indicated by 9, in the front side, of a transverse length L2 shorter than the transverse length L1 of the rear region. Numeral 10 indicates an intermediate region having a pair opposite of side borders adjacent 11, 11′ to said rear and front regions 6, 9. When in use, the attachment of the pair of attachments tabs 7, 7′ of the rear region to the front region may be through adhesive means, mechanical means and the like, and forms a circumference which adjusts radially to the user's body as mentioned above.

The side borders 11, 11′ are provided along a region of their extension with respective elastics 12, 12′, for adjusting the diaper to a groin region of the user's body, avoiding fluid leakage. In the intermediate region there is the absorbent core 5 disposed having a reduced thickness, in order to provide a comfortable use.

Conventionally, the attachment of the diaper to the body can be assured by several means, for example the attachments tabs 7, 7′ are applied to a front side at points adjacent to the front region 9, although pressure buttons, hook and loop fastener (VELCRO) and the like may be employed, the employed means being only a matter of convenience and not departing from the scope of the invention for the claimed disposable hygienic absorbent article.

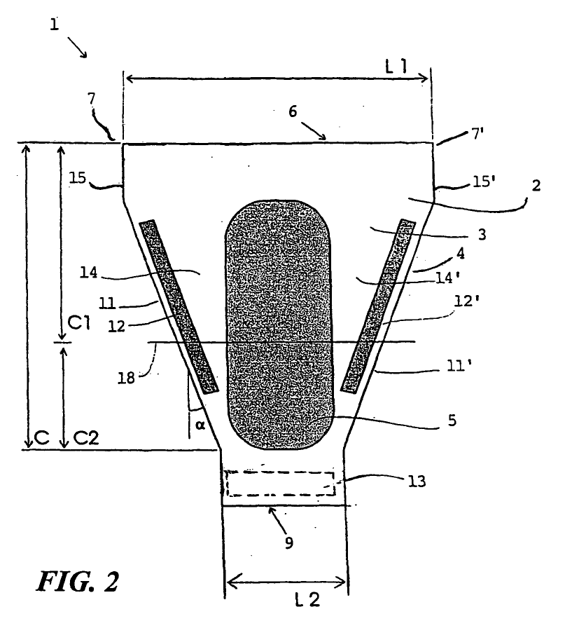

In a second embodiment shown in FIG. 2 the same reference numerals of FIG. 1 indicate identical components or those presenting a different form but an equivalent function. The essential aspects for accomplishing the invention are however maintained, only a few shape modifications being introduced and keeping both embodiments linked by the same inventive concept.

In the such second embodiment, the side borders 11, 11′ continue straight and parallel to each other in the direction of the rear region 6 along an adjacent portions indicated by 15, 15′, forming with said rear region a respective straight angle and the attachment tabs 7, 7′. The transverse length L1 is determined by the maximum opening ends of the side borders 11, 11′ or the distance between parallel adjacent portions 15, 15′. Numeral 13 indicates in dashed lines an area in the front region destined for attachment of the tabs 7, 7′. The absorbent core 5 shows a different shape and, in fact, the absorbent core 5 may assume any shape, new or already known in the state of the art.

FIGS. 1 and 2 diapers reveal a deviation from a long established development line, which admits compulsory loss of material due to curved cuts in the frame in order to secure a satisfactory anatomical fit with maximal reduction of leakage.

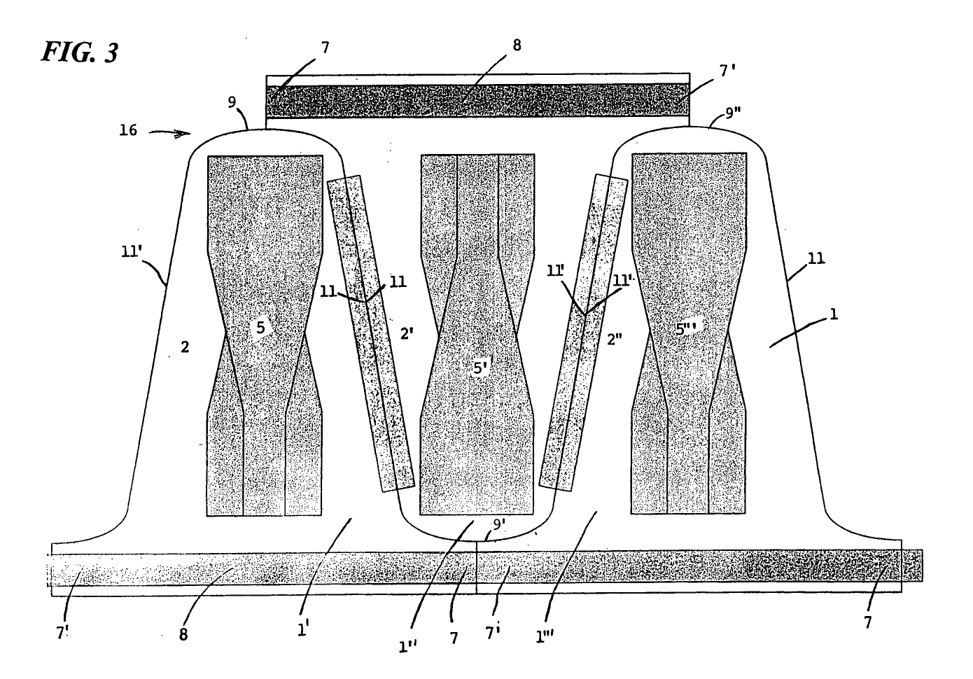

Effectively, as shown in FIGS. 1 and 2, the side borders 11 and 11′ are substantially straight, of definite slope at a slope angle indicated by, as measured between the respective side border 11, 11′ and a longitudinal symmetry axis of the absorbent article. The both side borders 11, 11′ converge mutually and symmetrically from the rear region 6 to the front region 9. All margins are non curved, except for a respective small curve between the side border 11, 11′ and attachment tabs 7, 7′, which forms a separation line for diapers disposed adjacent to each other over a continuous multilayer strip, as described in the following with reference to FIG. 3.

The slope angle may vary from 5 to 75 in relation to the longitudinal symmetry axis of the diaper 1, it preferably being 20. Close to each side border 11, 11′ the diaper 1 presents wall regions 14, 14′ adjacent to the absorbent core 5. The role of the wall regions 14, 14′ is to prevent side leakage, aided by said elastics stripes 12, 12′ shown as rectangular stripes and parallel to the already described side borders 11, 11′. In use, such distribution creates an imaginary fold line 18 substantially perpendicular to the longitudinal length C.

In order to provide an ideal protection situation against leakage, the ratio of the longer transverse length L1 to the shorter transverse length L2 greater than 2 is used, preferably ration L1 to L2 should be 3. Additionally, the ratio of the longitudinal length C to the longer transverse length L1 should be between 0.6 and 1.3, it preferably being 1.0. It should be noted that such ratios are obtained with the disposable hygienic absorbent article in an open position as shown in FIG. 1.

Another advantage provided by the present invention is its implementation by simple and economical steps and devices, with few manufacturing process modifications necessary. A manufacturing process consists of a first step of feeding a first continuous sheet of the fluid impermeable layer, whereupon a pre-molded the absorbent core is placed and anchored by adhesives. To the fluid impermeable layer sides borders elastic materials can be applied to improve the adjustment of the product to the user's body. A next step consists of placing the fluid permeable layer also structured by means of adhesive placement. In a structured “sandwich” the products are disposed on a mantle transversally and a cutting is done in a periodical fashion, the very same cutting lines forming the main body borders, with a final result of an compulsory loss reduced to zero.

FIG. 3 represents a region 16 of said first continuous sheet of multilayered material including a transversal disposition of three diapers 1′, 1″ and 1′″, side by side and adjacent to each other.

Just as the rest of the first continuous sheet of multilayer material, region 16 is part of said structured “sandwich” comprising the fluid permeable layer 3, the fluid impermeable layer 4 and the absorbent core 5 of substantially planar shaped disposed in-between.

A zero compulsory loss is evident in FIG. 3. A pair of side borders 8 in region 16 of the first continuous sheet defines the elastic stripe 8 on FIG. 1 diaper. A cutting line has a periodical wave shape along region 16 substantially shaped as a saw-tooth, whose oblique sides are the side borders 11 and 11′ of adjacent main bodies 2, 2′ and 2″ on each respective side of said cutting line. Inflection regions 9, 9′ and 9″ of the cutting line form a long curve, which is externally shared by the attachment tabs 7, 7′ of respective diapers 1 and 1″ and internally encloses an anterior distal region. As may be observed in the present embodiment, although the distal end of each adjacent diaper does not dispense the first continuous sheet borders, the compulsory loss of material of conventional processes is absent.

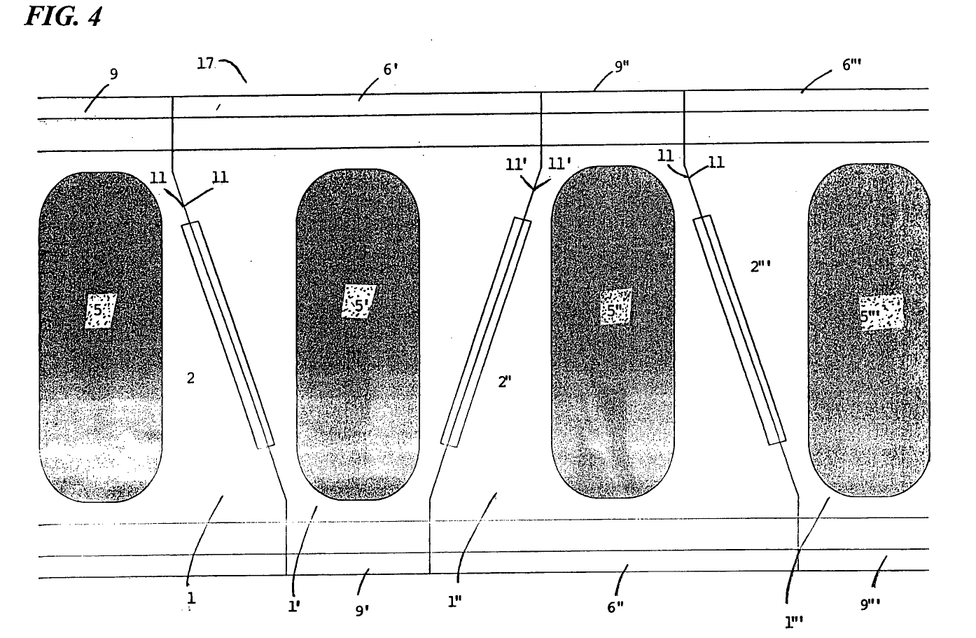

FIG. 4, analogously to FIG. 3, shows a region 17 of another possible multilayer material continuous sheet, which also includes a transversal disposition of four diapers 1, 1′, 1″ and 1′″ of FIG. 2, side by side and adjacent to each other.

In such case, the sectioning of adjacent main bodies 2, 2′, 2″ and 2′″ can be made by a non curved straight sided cutting line, such as a substantially square wave, or by the arrangement of a pair of parallel cutting cylinders with aligned axes, conveniently separated for passing the multilayer material continuous sheet 17 therethrough. The basic distinction from the transversal disposition of FIG. 2 diapers resides in the fact that the pair of borders of the multilayer material continuous sheet 17 define respectively opposed the regions 6 and 9 of main bodies 2, 2′, 2″ and 21″. In such second embodiment it also observed the absence of the characteristic compulsory loss of material found in conventional diapers and absorbents.

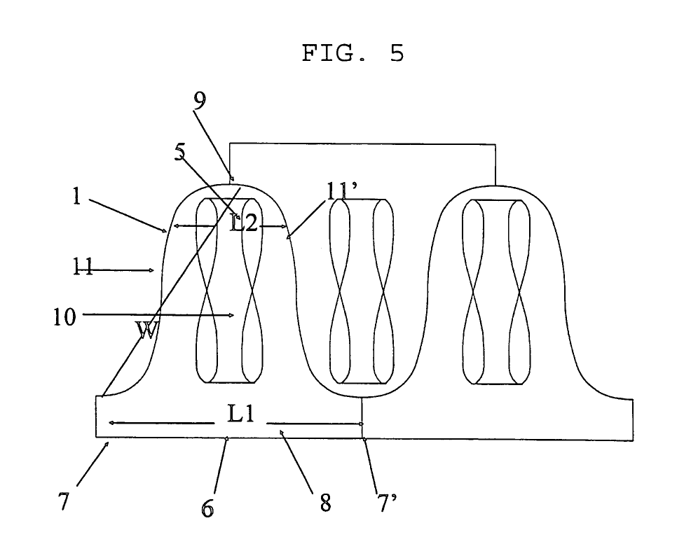

Referring now to FIG. 5, there is shown another embodiment of the invention, wherein the same reference numerals of FIG. 1 indicate identical components or those presenting a different form but an equivalent function. The essential aspects for accomplishing the invention are however maintained, only a few shape modifications being introduced and keeping both embodiments linked by the same inventive concept. As illustrated therein, the diaper 1 has a main body 2, which consists of a fluid permeable layer (not shown) adapted to contact the user' body and a fluid impermeable layer (not shown). Between the fluid permeable layer and the fluid impermeable layer there is disposed an absorbent core 5 having a substantially planar shape. The diaper has a rear region 6 having a transverse length L1. The rear region 6 may be formed from or may be provided with an elastic stripe 8. The rear region 6 ends in a pair of attachment tabs 7 and 7′ forming a diaper's waist for adjustment to the user's waist in use. The transverse length L1 of the rear region in the above mentioned example is obtained through the main body 2. However, the same transverse length L1 may be obtained through placing other additional materials, such as adhesive tapes and graft materials known in the state of the art by the term “extensions”.

Distally to the rear region 6 there is a front region indicated by 9, in the front side, of a transverse length L2 shorter than the transverse length L1 of the rear region. When in use, the attachment of the pair of attachments tabs 7, 7′ of the rear region to the front region may be through adhesive means, mechanical means and the like, and forms a circumference which adjusts radially to the user's body as mentioned above.

The disposable diaper 1 is made in a unitary piece, having a geometrical shape substantially of a truncated cone section with an upwards base and a longitudinal length C wherein the sides of the truncated cone are arcuate. The disposable diaper 1 has an intermediate region 10 having a pair opposite of side borders adjacent 11, 11′, the intermediate region being located between the rear and front regions 6, 9. Each side border 11, 11′ has a length W that extends from the rear region 6 to the front region 9. The side borders 11, 11′ of the disposable hygienic absorbent article have a longitudinal length and converge mutually and symmetrically at least once from the rear region to the front region along at least one half of their longitudinal length W. In a preferred embodiment, each respective side border 11 is a mirror image of the opposite side border 11′. In addition, when a side border 11 is curved, as shown in FIG. 5, the side border along a first one half of the longitudinal length W is a mirror image of the side border along the second one half of the longitudinal length W.

In such manner, the present invention provides a disposable hygienic absorbent having a longer length in the rear region, allowing an excellent liquid retention and the like. It also provides an excellent adjustment in the front region, increasing the comfort during use.

Having now described a preferred embodiment, it should be understood that the scope of the present invention encloses other possible variations, being limited only by the content of the following claims, including possible equivalents.

1. A disposable hygienic absorbent article comprising a fluid permeable material layer, a fluid impermeable layer, an absorbent core disposed between said fluid permeable layer and the impermeable layer, the disposable hygienic absorbent article having a longitudinal symmetry axis, a rear region having a transverse length adapted to encircle a user's waist in use and an opposite front region having a transverse length that is less than the transverse length of the rear region, either the front region or the rear region having an attachment means adapted to join the front region to the rear region around the user's waist in use, an intermediate region adjacent to said rear region and said front region, the intermediate region having a pair of opposite side borders, the side borders having a longitudinal length, wherein the side borders converge mutually and symmetrically at least once from the rear region to the front region and along at least one half of their longitudinal length.

2. Hygienic absorbent article according to claim 1, wherein the longitudinal length of the side borders includes a first half and a second half and wherein the first half is symmetrical with the second half.

3. Hygienic absorbent article, according to claim 1, wherein the side borders converge along a substantially straight line defining a slope angle, as measured between one side border and the longitudinal symmetry axis of the absorbent article and wherein the slope angle is between 5° and 75°.

4. Hygienic absorbent article, according to claim 1, wherein said hygienic absorbent article is a diaper, incontinence pad and the like.

5. Hygienic absorbent article, according to claim 1, wherein in use said intermediate region is adjacent a groin region of the user's body in use.

6. Hygienic absorbent article, according to claim 1, wherein a ratio of the transverse length of the rear region to the transverse length of the front region is greater than 2 when the hygienic absorbent article is in an opened position.

7. Hygienic absorbent article, according to claim 6, wherein the ratio of the transverse length of the rear region to the transverse length of the front region is 3 when the hygienic absorbent article is in an opened position.

8. Hygienic absorbent article, according to claim 1, wherein the article has a length as measured between the rear region and the front region along the longitudinal symmetry axis and wherein a ratio of the length of the article to the transverse length of the rear region is between 0.6 and 1.3 when the hygienic absorbent article is in an opened position.

9. Hygienic absorbent article, according to claim 8, wherein the ratio of the length of the article to the transverse length of the rear region is 1.0.

10. Hygienic absorbent article, according to claim 3, wherein the slope angle is 20°.
